(19) United States
(12) Patent Application Publication
(10) Pub. No.: US 2022/0202432 A1
(43) Pub. Date: Jun. 30, 2022

(54) Fecal Impact Remover Tool (F-Cal Tool)

(71) Applicants: Lerrlyn Nelson, Detroit, MI (US); Patrick Munson, Waves, NC (US)

(72) Inventors: Lerrlyn Nelson, Detroit, MI (US); Patrick Munson, Waves, NC (US)

(21) Appl. No.: 17/457,656

(22) Filed: Dec. 4, 2021

(51) Int. Cl. A61B 17/22

(52) U.S. Cl. CPC A61B 17/22031; A61B 2017/22079; A61B 2017/22034; A61B 2017/22037

(57) ABSTRACT

A safe, practical, method for physical removal of a fecal impaction. This device empowers the clinical community to treat persons' who suffers from the painful condition in a more compassionate way without the fear of causing more harm to the patient.

### TECHNICAL FIELD

A safe, practical, method for physical removal of a fecal impaction. This device empowers the clinical community to treat persons' who suffers from the painful condition in a more compassionate way without the fear of causing more harm to the patient.

### BACKGROUND OF THE INVENTION

### A. Field of the Invention

This invention relates primarily to the problem of fecal impaction is inadequately deli with initially by primary care often over the phone suggesting mild laxatives. Since the problem is not in the colon but in the rectum this rarely works. In a typical situation the patient presents to an emergency setting and is given an IV saline because many times the patient is dehydrated. This is neither the cause of the problem nor its solution it is rather an effect of the pain the patient has been enduring. Labs are often drawn ostensibly to rule out other problems. In some advance cases the patient is suffering electrolyte imbalance or ammonia toxicity but this is due not to the primary problem but to the fact that it has gone on far too long. The next step is abdominal X-rays are taken or a CT scan of the abdomen is completed to rule out higher colonic obstruction. Although these procedures are conducted none of these measures directly address the problem of fecal impaction and have become common due to the need to put off actually dis-impacting the patient.

### BACKGROUND ART

All patients presenting with a medical problem should be treated equally, humanely and with genuine compassion. Problems that are difficult, embarrassing, or painful should not take a second seat in the process of medical care. The individuals who have trained and who put their hearts into delivering compassionate care need and deserve compassionate tools to perform their job. For these reasons this device takes a major step in treating the patient with a compassionate tool. Fecal impaction is a common and difficult problem encountered by many persons that have anal muscle weakness due to age or illness. At present the only option to address this problem is emergency medical care. Another reason Fecal impaction is because early impactions are generally not treated until pain is severe which bring the patient to the emergency room. Even upon presentation to an emergency department the treatment options are limited to laxatives given orally which do not generally solve the problem and may exacerbate pain as motility is not the current priority. The other option the medical community have to treat this fecal impaction is digital removal, which is painful, time-consuming for medical personnel and often avoided until absolutely necessary.

As mentioned above it has been factually proven and well known, that Fecal Impact is extremely difficult for many who suffer from this challenge. Although there are existing treatment for treating various blockages within the human body for this condition none specifically has been designed to function in this manner for this purpose. In order to assist in overcoming this problem, some treatments have been designed for the ease of various blockage treatment within the human body they do not relieve the problem this device is intended to address in this manner. For example:

U.S. Pat. No. 05/828,397, entitled “METHOD FOR TREATING KIDNEY STONES.” (Imondi, et al.) discloses a Water soluble and colloidially water soluble polymers of carboxylic acid-containing monomers useful for treating kidney stones are disclosed.

U.S. Patent Pub. No. 10/857,326, entitled “PLU OMARY EMBOLISM APPARATUS” (Kelly) discloses as an apparatus that includes a first catheter having a proximal end and a distal end. The apparatus also includes a first tubular housing having a proximal end and a distal end. The first tubular housing is coupled to the first catheter proximal to at least one first outlet. The apparatus also includes one or more pressure sensors coupled to the distal end of the first tubular housing. The apparatus also includes a second catheter having a proximal end and a distal end. The distal end of the second catheter is configured to be positioned substantially within a second tubular housing coupled to one or more of the first catheter and the first tubular housing, when the second catheter is in a first position.

U.S. Pat. No. 11,185,625, B2 (Hassidov et al.) entitled “COLON CLEANING SYSTEM WITH AUTOMATIC SELF-PURGING FEATURES” a Systems and methods for cleaning a colon or other portion of an intestine include optional use of sensors to detect conditions of blockage of flow of materials within an evacuation channel used to remove fecal material from the body; and devices and methods for purging such blockages from the evacuation channel.

Story:

Fecal impaction is a common and difficult problem encountered by many persons with anal muscle weakness due to age or illness. At present the only option to address this problem is emergency medical care. Fecal impaction is a common problem and for this reason early impactions are generally not treated until pain is severe which bring the patient to the emergency room. Even upon presentation to an emergency department the treatment options are limited to laxatives given orally which do not generally solve the problem and may exacerbate pain as motility is not the current priority. Another option is “Digital removal”, which is painful, time-consuming for medical personal and often avoided until absolutely necessary. These are just two reasons why “Fecal Impaction Remover” was designed and invented

### BRIEF DESCRIPTION OF THE DRAWINGS

FIG. 1 is a perspective view of the Fecal Impact Remover tool (F-Cal) showing the fully assembled position of the present invention Collection Bulb and Toomey Port.

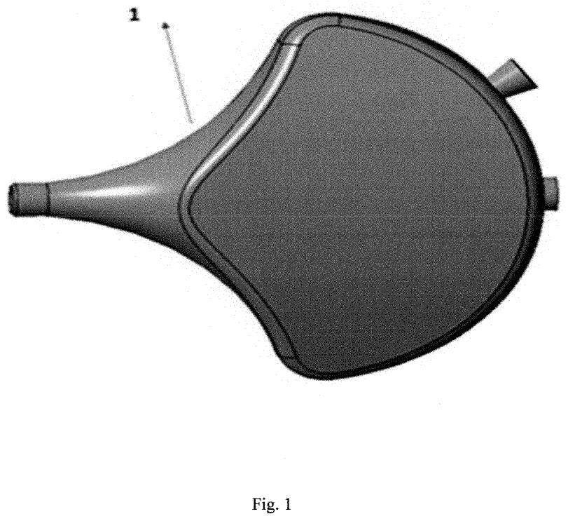

FIG. 2 is a perspective view of the Fecal Impact Remover tool (F-Cal) showing the in-side perspective of a fully assembled position of the present invention Collection Bulb and Toomey Port.

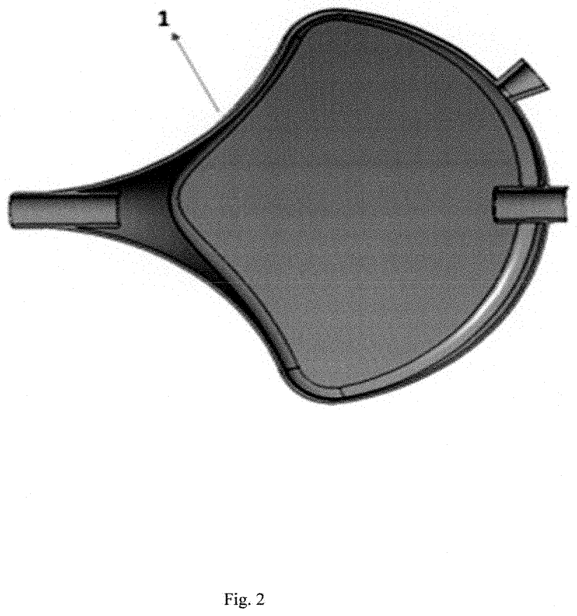

FIG. 3 is a perspective view of the Fecal Impact Remover tool (F-Cal) showing the fully assembled position of the present invention with Collection Bulb, Insertion Trocar, and Toomey Port

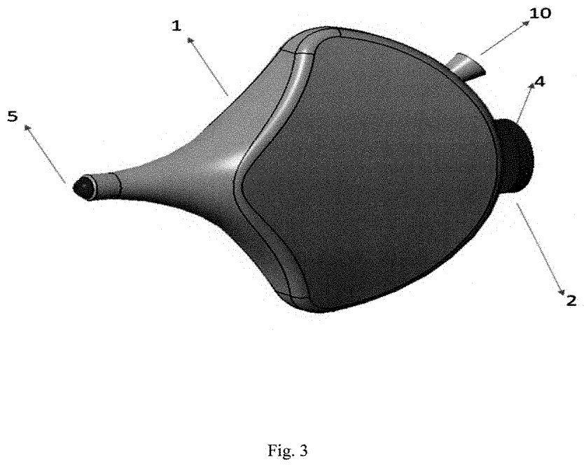

FIG. 4 is a perspective view of the Fecal Impact Remover tool (F-Cal) showing the fully assembled position of the present invention Collection Bulb, but with-out displaying the Insertion Trocar and Insertion Trocar Handle but including the Screw Trocar with Screw Trocar Handel and Toomey Port.

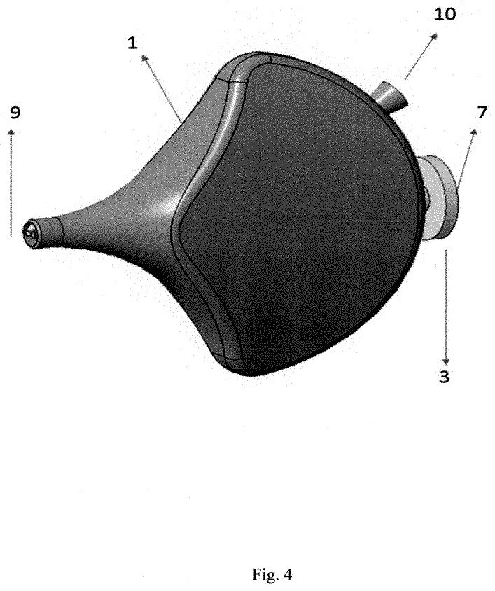

FIG. 5 is a perspective view of the Fecal Impact Remover tool (F-Cal) showing the fully assembled position of the Insertion Trocar Handel, Insertion Trocar Bullet Introducer Tip, Insertion Trocar Suppository Port and how it is positions within the tip of Collection Bulb.

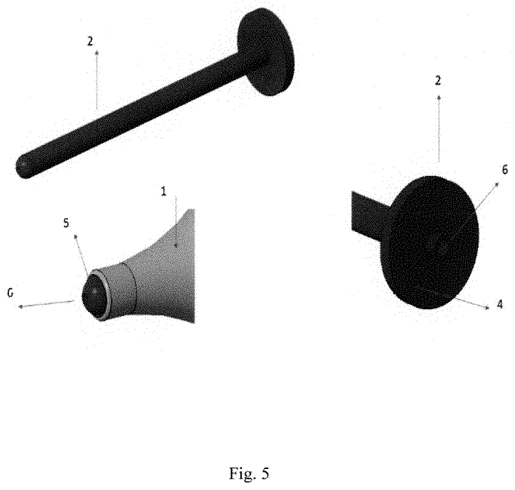

FIG. 6 is a perspective view of the Fecal Impact Remover tool (F-Cal) showing the fully assembled position of the Screw Trocar with Screw Trocar Handel the Screw Trocar Irrigation Port, Screw Trocar Screw Recess and how it is positions within the tip of Collection Bulb.

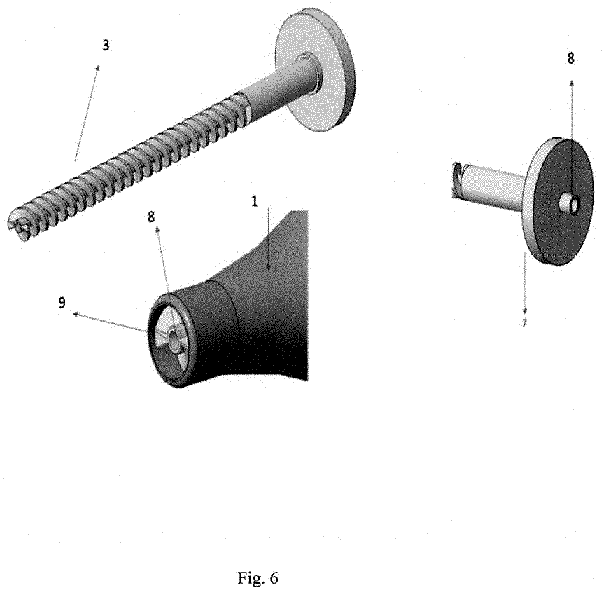

FIG. 7 is a perspective view of the Fecal Impact Remover tool (F-Cal) showing the in-side perspective of a fully assembled position of the present invention with-in the Collection Bulb showing the Insertion Trocar, Screw Trocar, Insertion Trocar Handel, Insertion Trocar Bullet Introducer Tip, Insertion Trocar Suppository Port and Toomey Port.

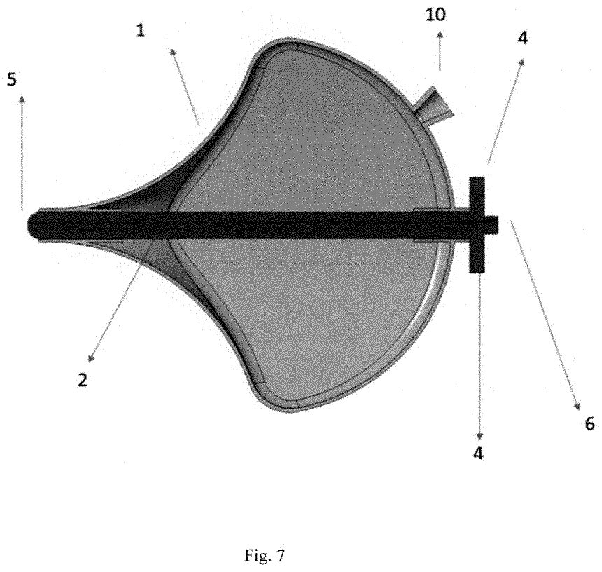

FIG. 8 is a perspective view of the Fecal Impact Remover tool (F-Cal) showing the in-side perspective of a fully assembled position of the present invention with-in the Collection Bulb showing the Screw Trocar, Screw Trocar Handel Screw Trocar Irrigation Port and Toomey Port.

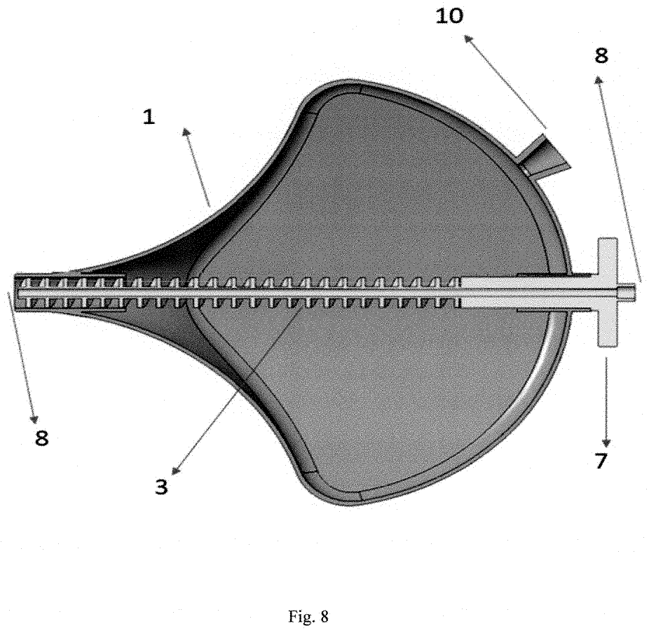

### REFERENCE LIST

Fecal Impact Remover Tool (F-Cal Tool)
1. Collection Bulb
2. Insertion Trocar
3. Screw Trocar
4. Insertion Trocar Handel
5. Insertion Trocar Bullet Introducer Tip
6. Insertion Trocar Suppository Port
7. Screw Trocar Handel
8. Screw Trocar Irrigation Port
9. Screw Trocar Screw Recess
10. Toomey Port

1. The Fecal Impact remover is a one-time use medical device which is designed to decrease fecal material content within the rectum thereby decreasing the pain cause by the fecal impaction in the rectal vault and allow its eventual resolution.

2. The medical device described in claim 1 consists of a housing called the collection bulb and two tracers, the insertion trocar and the screw trocar.

3. The medical device described in claim 2 has a central channel that is divided into two parts. The exterior part directs all trocars through the center of the device and into the internal channel. The internal channel terminates at the collection bulb.

4. The medical device described in claim 3, the small end of the device has a logarithmic curve to meet the larger oval exterior part of the device thus preventing the device from being inserted too far.

5. The medical device described in claim 4 is first used with the insertion trocar which has a bullet shaped end occupying the central channel allowing and promoting easy penetration of the rectal sphincter.

6. The medical device described in claim 5 once inserted into the rectal sphincter the bullet trocar is removed and a screw shaped trocar will be introduced throughout the device's central channel and thus through the rectal sphincter.

7. The medical device described in claim 6 the distal end of the screw boring device is recessed in shape within the central channel such that it does not protrude from the end to ensure it does not touch the rectal wall.

8. The medical device described in claim 7 In addition the cutting tip of the screw device is not sharp but rounded in a way to allow penetration of the impaction without danger of injury to the rectal wall.

9. The medical device described in claim 8 the screw shaped trocar can be turned in a twisting motion by its handle at the exterior end of the central channel through the device.

10. The medical device described in claim 9 In addition to the screw boring end the screw trocar device has a central channel to deliver irrigation fluid from a port at the exterior handle to the screw tip.

11. The medical device described in claim 10 the irrigation fluid can be tap water but may have glycerin added to the fluid and may be warmed.

12. The medical device described in claim 11 has bulb like area at the end of the internal channel where the stool that has been removed by the screw trocar is collected.

13. The medical device described in claim 12 is sealed at the outer most junction of the central channel with the collection bulb reducing odor and retaining stool inside the device.

14. The medical device described in claim 13 has a drain bag that may be attached to the impaction remover by a one-way valve to allow a greater volume of irrigation fluid and stool to be accommodated without opening the sealed device.

15. The medical device described in claim 14 by turning the screw and irrigating bores a hole in the center of the impaction delivering this stool and irrigation fluid to the collection bulb.

16. The medical device described in claim 15 removes the center of the impaction without increasing the volume of contents within the rectal vault and thus does not increase pain.

17. The medical device described in claim 16 a collection bulb which is sealed sufficiently to allow a hand vacuum pump such as a Toomey syringe to apply vacuum to the bulb as needed if the substance of the impaction is firm.

18. The medical device described in claim 17 the bullet end of the insertion trocar can be removed presenting a holder for a standard glycerin suppository.

19. The medical device described in claim 18 has a plunging stylus in the center of the insertion trocar to facilitate removal of the insertion bullet and to eject the suppository into the impaction after a core has been removed.

20. The medical device described in claim 19 is a single use tool that will be packaged sterile and may be supplied with extension
